(19) United States
(12) Patent Application Publication
(10) Pub. No.: US 2017/0121778 A1
(43) Pub. Date: May 4, 2017

(54) E2F4 SIGNATURE FOR USE IN DIAGNOSING AND TREATING BREAST AND BLADDER CANCER

(71) Applicant: Trustees of Dartmouth College, Hanover, NH (US)

(72) Inventor: Chao Cheng, Lebanon, NH (US)

(73) Assignee: Trustees of Dartmouth College, Hanover, NH (US)

(21) Appl. No.: 15/314,579

(22) PCT Filed: Jun. 19, 2015
(86) PCT No.: PCT/US2015/036567

§ 371 Date: Nov. 29, 2016

Related U.S. Application Data
(60) Provisional application No. 62/030,884, filed on Jul. 30, 2014.
(60) Provisional application No. 62/154,226, filed on Apr. 29, 2015.

(51) Int. Cl. C12Q 1/68; A61K 39/04

(52) U.S. Cl. CPC C12Q 1/6886; A61K 39/04; C12Q 2600/118; C12Q 2600/158

(57) ABSTRACT

Methods for treating breast and bladder cancer patients based upon E2F4 regulatory activity as a predictor of relapse of a patient with estrogen receptor positive breast cancer and in bladder cancer stratification are provided. The methods involve determining gene expression profiles for substances linked to activity of E2F4 in cancer cells and tissue. Signature gene expression profiles are provided for identifying breast cancer samples and bladder cancer samples that would be responsive to certain therapies and also provide for prognosis based on these profiles.

### BACKGROUND

Cancer prognosis and treatment plans rely on a collection of clinicopathological variables that stratify cancers outcomes by stage, grade, responsiveness to adjuvant therapy, and so on. Despite stratification, cancer's enormous heterogeneity has made precise outcome prediction elusive and the selection of the optimal treatment for each patient a difficult and uncertain choice. Over the past two decades, advances in molecular biology have allowed molecular signatures to become increasingly obtainable (Liotta & Petricoin (2000) Nat. Rev. Genet. 1:48-56) and incorporated into determining cancer prognosis and treatment (Ginsburg & Willard (2009) Transl. Res. 154:277-87). For some cancer types, like breast cancer, gene expression signatures are now routinely used prognostically, with many research groups having identified signatures that predict cancer outcome or consider if patients will benefit from adjuvant therapy following surgical resection (Van't Veer, et al. (2002) Nature 415:530-6; van der Vijver, et al. (2002) N. Engl. J. Med. 347:1999-2009; Wang, et al. (2005) Lancet 365:671-9; Sotiriou, et al. (2006) J. Natl. Cancer Inst. 98:262-72; Miller, et al. (2005) Proc. Natl. Acad. Sci. USA 102:13550-5; Pawitan, et al. (2005) Breast Cancer Res. 7:R953-64; Hornberger, et al. (2012) J. Natl. Cancer Inst. 104:1068-79). Even with gene expression signatures' successes in cancer outcome prediction, improvement is possible, as the majority of these signatures are applicable only to early stage cancers without lymph node metastasis or even previous chemotherapy. As cancer is fundamentally a disease of genetic dysregulation, specifically analyzing a tumor's regulatory actors, such as transcription factors, may provide additional prognostic insight (Eckhoff, et al. (2013) J. Cancer Res. Clin. Oncol. 139:1673-80; Haq & Fisher (2011) J. Clin. Oncol. 29:3474-82), since transcription factors are relatively universal among different cell lines when compared to the tissue-specific gene clusters from which most gene signatures are made.

Transcription factors are proteins that relay cellular signals to their target genes by binding to the DNA regulatory sequences of these genes and modulating their transcription (Mitchel & Tjian (1989) Science 245:371-8). They play major roles in many diverse cellular processes (Helin (1998) Curr. Opin. Genet. Dev. 8:28-35; Barkett & Gilmore (1999) Oncogene 18:6910-24; Ogino, et al. (2012) Dev. Biol. 363:333-47; Kako & Ishida (1998) Neurosci. Res. 31:257-64; Sanchez-Tillo, et al. (2012) Cell. Mol. Life Sci. 69:3429-56). Unsurprisingly, aberrant expression or mutation of transcription factors or of their upstream signaling proteins has been implicated in an array of human diseases, including cancer (Darnell, Jr. (2002) Nat. Rev. Cancer 2:740-9; Suva, et al. (2013) Science 339:1567-70; Nebert (2002) Toxicology 181-182:131-41).

While differences in the transcriptional expression level of a transcription factor do not necessarily correspond to differences in its regulatory activity, differences in the expression levels of a transcription factor's target genes do (Cheng, et al. (2007) BMC Bioinformatics 8:452; Rhodes, et al. (2005) Nat. Genet. 37:579-83; Cheng & Li (2008) BMC Genomics 9:116). An algorithm, called REACTIN (REgulatory ACTivity INference), has been developed to make this inference of a transcription factor's regulatory activity from the expression of its target genes (Zhu, et al. (2013) BMC Genomics 14:504). REACTIN can calculate the activity level of a transcription factor on each individual sample in a given dataset. By calculating these levels and generating individual Regulatory Activity Scores (iRASs) for a given transcription factor and sample, REACTIN reveals a given transcription factor's activity level for each individual sample relative to all others in a dataset, thereby enabling the incorporation of a transcription factor's activity level into regression-based analyses. For example, by combining these iRAS transcription factor activity levels with survival data, Cox proportional hazard (PH) models can be employed to examine how transcription factor activity levels correlate with survival outcomes.

### SUMMARY OF THE INVENTION

This invention is a method of administering an aggressive breast cancer treatment (a) providing a ER+ breast tumor tissue sample from a patient; (b) measuring the expression of genes regulated by transcription factor E2F4 in the ER+ breast tumor tissue sample; (c) inferring changes in transcription factor E2F4 activity in the ER+ breast tumor tissue sample using the measured expression in (b); (d) comparing the inferred changes in transcription factor E2F4 activity in the ER+ breast tumor tissue sample to transcription factor E2F4 activity in a reference sample; and (e) administering an aggressive breast cancer treatment to the patient when the ER+ breast tumor tissue sample has higher transcription factor E2F4 activity than in the reference sample. In one embodiment the expression of genes regulated by transcription factor E2F4 is performed by microarray analysis with probes specific to the genes regulated by transcription factor E2F4. In another embodiment, the genes regulated by transcription factor E2F4 are listed in Table 1. In a further embodiment, the aggressive breast cancer treatment comprises chemotherapy, radiation or a combination thereof.

This invention is also a method of administering intravesical BCG immunotherapy by (a) providing a non-muscle invasive bladder cancer sample from a patient; (b) measuring the expression of genes regulated by transcription factor E2F4 in the non-muscle invasive bladder cancer sample; (c) inferring changes in transcription factor E2F4 activity in the non-muscle invasive bladder cancer sample using the measured expression in (b); (d) comparing the inferred changes in transcription factor E2F4 activity in the non-muscle invasive bladder cancer sample to transcription factor E2F4 activity in a reference sample; and (e) administering intravesical BCG immunotherapy to the patient when the non-muscle invasive bladder cancer sample has higher transcription factor E2F4 activity than in the reference sample. In one embodiment, the expression of genes regulated by transcription factor E2F4 is performed by microarray analysis with probes specific to the genes regulated by transcription factor E2F4. In another embodiment, the genes regulated by transcription factor E2F4 are listed in Table 1.

### BRIEF DESCRIPTION OF THE DRAWINGS

FIG. 1 shows E2F4 activity and expression levels throughout the cell cycle in HeLa S3 cells. Activity was calculated as RAS, the regulatory activity score, and expression was calculated in log ratio from cDNA array. The inferred E2F4 activity derived from RAS (solid black line), but not the E2F4 expression level (dashed line), was significantly periodic during the cell cycle.

FIG. 2 demonstrates that patients with positive E2F4 scores show significantly shorter survival times than those with negative E2F4 scores. Vertical hash marks indicate points of censored data. Results were derived from the Vijver dataset with overall survival (os) as the endpoint.

FIG. 3 shows a Kaplan Meier plot of pooled, un-stratified breast cancer datasets. As with the un-pooled results, positive E2F4 scores show shorter survival times than those with negative E2F4 scores across all datasets (p-value=1.43e-21, log-rank test). RFS: relapse-free survival.

FIG. 4 shows the application of the E2F4 signature for predicting patient survival times in estrogen receptor (ER) histological subtypes. Note that E2F4 signature is effective in ER+ but not in ER− samples. RFS: relapse-free survival.

FIG. 5 shows the distribution of E2F4 scores in primary bladder tumor samples.

FIGS. 6A, 6B and 6C show that the E2F4 program is predictive of the efficacy of intravesical BCG immunotherapy in NMIBC. The survival curves of intravesical therapy treated and untreated groups were compared in all samples (FIG. 6A), and samples with E2F4>0 (FIG. 6B) and E2F4<0 (FIG. 6C). IVT: intravesical BCG immunotherapy; PFS: progression-free survival. Number of samples are in parenthesis.

### DETAILED DESCRIPTION OF THE INVENTION

It has now been found that E2F4 regulatory activity is of use as a predictor of relapse of a patient with estrogen receptor positive (ER+) breast cancer and in bladder cancer stratification. Using E2F4 regulatory activity analysis, breast cancer patients at a high or low risk of relapsing can now be identified and, if found to be at high risk, be administered an aggressive breast cancer treatment regime, e.g., additional chemotherapy and/or radiation. The method can complement ONCOTYPE DX, which is currently in clinical use for identifying high, intermediate and low risk subjects, but does not stratify those subjects in the intermediate risk group that could benefit from treatment. Similarly, using the instant invention, subjects with non-muscle invasive bladder cancer and exhibiting a positive E2F4 score can be identified and administered intravesical BCG immunotherapy.

Accordingly, in one embodiment, the present invention provides a method for administering an aggressive breast cancer treatment by providing a ER+ breast tumor tissue sample from a patient; measuring the expression of genes regulated by transcription factor E2F4; inferring changes in transcription factor E2F4 activity in the ER+breast tumor tissue sample using the expression data; comparing the inferred transcription factor E2F4 activity in the sample to E2F4 activity in a reference sample; and administering an aggressive breast cancer treatment to the patient when the ER+ breast tumor tissue sample has higher transcription factor E2F4 activity than in the reference sample.

In another embodiment, the present invention provides a method for administering intravesical BCG immunotherapy by providing a non-muscle invasive bladder cancer sample from a patient; measuring the expression of genes regulated by transcription factor E2F4 in the non-muscle invasive bladder cancer sample; inferring changes in transcription factor E2F4 activity in the non-muscle invasive bladder cancer sample using the expression data; comparing the inferred changes in transcription factor E2F4 activity in the non-muscle invasive bladder cancer sample to transcription factor E2F4 activity in a reference sample; and administering intravesical BCG immunotherapy to the patient when the non-muscle invasive bladder cancer sample has higher transcription factor E2F4 activity than in the reference sample.

In accordance with the methods of this invention, only patients that would benefit, e.g., by increased survival and/or reduced cancer recurrence, are treated thereby reducing unnecessary and/or costly treatments.

Breast Cancer. Breast tumors often, but do not always, have hormone receptors, more particularly estrogen and progesterone receptors, that can be detected in tissue samples obtained by biopsy prior to surgery or in tissue samples obtained during surgery. A tumor in which estrogen receptors (ER) are identified is said to be estrogen receptor positive (ER+), and one lacking ER is said to be estrogen receptor negative (ER−). Likewise, tumors can be progesterone receptor positive (PR+) or negative (PR−). Any assay known in the art for detection of estrogen receptors can be used. Assay methods include, without limitation, ligand binding assays, immunohistochemical assays (including immunocytochemical assays) and combinations thereof. Reference may be made, for example, to Graham, et al. (1999) Am. J. Vet. Res. 60:627-630; Heubner, et al. (1986) Cancer Res. 46(8 suppl.):4291s-4295s and Harvey et al. (1999) J. Clin. Oncol. 17:1474-1481.

ER+ breast cancer is often treatable with drugs that bind more or less selectively to ER. Such drugs partially or completely prevent estrogen from binding to ER and thereby modulate a cascade of events leading to cell proliferation and tumor growth. Tamoxifen was the first, and is still most widely used, of a class of such drugs known as selective estrogen receptor modulators (SERMs). SERMs are useful not only in palliative treatment of ER+breast cancer but have marked prophylactic utility in healthy subjects at high risk of developing breast cancer, for example subjects having family history of the disease or a previous finding of atypical hyperplasia or in situ carcinoma in a breast tissue biopsy. Another SERM, raloxifene, has likewise been found to have prophylactic value in reducing incidence of invasive breast cancer, at least in postmenopausal women (Cummings, et al. (1999) JAMA 281(23):2189-2197). Another approach to treatment of estrogen-sensitive breast cancer is to reduce the level of estrogen circulating in the patient and thereby reduce the amount of estrogen available for binding to ER in breast tissue. This can be accomplished, for example, by inhibition of aromatase, an enzyme involved in biosynthesis of estrogen from androgens. Aromatase inhibitors such as anastrozole, exemestane and letrozole are available for treatment of ER+ invasive breast cancer. In accordance with the present invention, an aggressive breast cancer treatment can include surgical intervention, chemotherapy with a given drug or drug combination as described herein, and/or radiation therapy.

Bladder Cancer. Urinary bladder (or bladder) cancer is one of the most common cancers worldwide, with the highest incidence in industrialized countries. Two main histological types of bladder cancer are the urothelial cell carcinomas (UCC) and the squamous cell carcinomas (SCC). The UCCs are the most prevalent in Western and industrialized countries and two third of the patients with UCC can be categorized into non-muscle invasive bladder cancer (NMIBC) and one third in muscle invasive bladder cancer (MIBC). In NMIBC, the disease is generally confined to the bladder mucosa (stage Ta, carcinoma in situ (CIS)) or bladder submucosa (stage T1). In MIBC, the patient has a tumor initially invading the detrusor muscle (stage T2), followed by the perivesical fat (stage T3) and the organs surrounding the bladder (stage T4). The management of NMIBC can include transurethral resection followed by adjuvant intravesical therapy with BCG (Bacillus Calmette Guerin), the most effective intravesical treatment, for high-risk patients (Kamat & Lamm (2001) Curr. Urol. Rep. 2:62-69); however, a significant number of patients fail treatment and require more aggressive intervention, such as radical cystectomy and/or chemotherapy. Therefore, the present invention can be used to identify those NMIBC patients likely to respond to BCG immunotherapy as well as those patients that may require more aggressive intervention.

E2F4 Signature. Members of the E2F family of transcriptional regulators functionally interact with the pocket protein transcription factors, p107, p130, and pRb. The nature of these interactions defines the transcriptional regulatory complexes as activators or repressors. These complexes regulate expression of a variety of genes, many of which are associated with cell cycle regulation (Nevins (1998) Cell Growth Differ. 9:585-93). The activating E2Fs, namely E2F1, E2F2, and E2F3a, promote the G₁-to-S phase transition during cell cycle progression (Wu, et al. (2001) Nature 414:457-62), interacting with the basal transcriptional machinery to enhance expression of cyclin E, DNA polymerase α, thymidine kinase, and other genes that advance the cell cycle (La Thangue (2003) Nat. Cell Biol. 5:587-9). In contrast, the repressing E2Fs, namely E2F3b, E2F4 and E2F5, have the ability to bind similar promoter regions to those bound by the activating E2Fs (Araki, et al. (2003) Oncogene 22:7632-41), but are simultaneously bound by pocket proteins pRb, p107, or p130, that physically prevent interaction with the transcriptional machinery (Dyson (1998) Genes Dev. 12:2245-62).

Genes regulated by E2F4, the expression of which are analyzed in accordance with the present invention, include, but are not limited to, one or more the genes listed in Table 1.

TABLE 1

| Gene | Description |
|---|---|
| NUDT2 | nudix (nucleoside diphosphate linked moiety X)- |
| | type motif 2 |
| CEP192 | centrosomal protein 192 kDa |
| ATAD2 | ATPase family, AAA domain containing 2 |
| MCM3 | minichromosome maintenance complex component 3 |
| CENPK | centromere protein K |
| SPC25 | SPC25, NDC80 kinetochore complex component, |
| | homolog (S. cerevisiae) |
| CDCA8 | cell division cycle associated 8 |
| GMNN | geminin, DNA replication inhibitor |
| MND1 | meiotic nuclear divisions 1 homolog |
| | (S. cerevisiae) |
| CDC6 | cell division cycle 6 homolog (S. cerevisiae) |
| E2F3 | E2F transcription factor 3 |
| SMC6 | structural maintenance of chromosomes 6 |
| CDCA3 | cell division cycle associated 3 |
| RAD54L | RAD54-like (S. cerevisiae) |
| MYBL2 | v-myb myeloblastosis viral oncogene homolog |
| | (avian)-like 2 |
| AP4M1 | adaptor-related protein complex 4, mu 1 subunit |
| BLM | Bloom syndrome, RecQ helicase-like |
| CASC5 | cancer susceptibility candidate 5 |
| MCM7 | minichromosome maintenance complex component 7 |
| RAD9B | RAD9 homolog B (S. pombe) |
| DTL | denticleless homolog (Drosophila) |
| NAP1L4 | nucleosome assembly protein 1-like 4 |
| CENPF | centromere protein F, 350/400 ka (mitosin) |
| RAD18 | RAD18 homolog (S. cerevisiae) |
| ZFPL1 | zinc finger protein-like 1 |
| KIF14 | kinesin family member 14 |
| NDC80 | NDC80 homolog, kinetochore complex component |
| | (S. cerevisiae) |
| UBE2S | ubiquitin-conjugating enzyme E2S |
| LRRC14 | leucine rich repeat containing 14 |
| GTSE1 | G-2 and S-phase expressed 1 |
| KIF23 | kinesin family member 23 |
| C1orf35 | chromosome 1 open reading frame 35 |
| CENPA | centromere protein A |
| C11orf10 | chromosome 11 open reading frame 10 |
| METTL4 | methyltransferase like 4 |
| SF3A2 | splicing factor 3a, subunit 2, 66 kDa |
| FEN1 | flap structure-specific endonuclease 1 |
| ASH2L | ash2 (absent, small, or homeotic)-like |
| | (Drosophila) |
| FAM76B | family with sequence similarity 76, member B |
| RCCD1 | RCC1 domain containing 1 |
| FBXO5 | F-box protein 5 |
| SIVA1 | SIVA1, apoptosis-inducing factor |
| ZNF688 | zinc finger protein 688; zinc finger protein 785 |
| EXO1 | exonuclease 1 |
| C18orf56 | chromosome 18 open reading frame 56 |
| ANLN | anillin, actin binding protein |
| KIF24 | kinesin family member 24 |
| GBA | glucosidase, beta; acid (includes |
| | glucosylceramidase) |
| SYCE2 | synaptonemal complex central element protein 2 |
| C19orf57 | chromosome 19 open reading frame 57 |
| DCLRE1B | DNA cross-link repair 1B (PSO2 homolog, |
| | S. cerevisiae) |
| NCAPG | non-SMC condensin I complex, subunit G |
| RAD51 | RAD51 homolog (RecA homolog, E. coli) |
| | (S. cerevisiae) |
| NCAPG2 | non-SMC condensin II complex, subunit G2 |
| C11orf82 | chromosome 11 open reading frame 82 |
| CDT1 | chromatin licensing and DNA replication factor 1 |
| EZH2 | enhancer of zeste homolog 2 (Drosophila) |
| KIAA1731 | KIAA1731 |
| OIP5 | Opa interacting protein 5 |
| IQGAP3 | IQ motif containing GTPase activating protein 3 |
| NCAPH | non-SMC condensin I complex, subunit H |
| SHC1 | SHC (Src homology 2 domain containing) |
| | transforming protein 1 |
| FAM111A | family with sequence similarity 111, member A |
| DGCR8 | DiGeorge syndrome critical region gene 8 |
| KIF18B | kinesin family member 18B |
| MLF1IP | MLF1 interacting protein |
| CKAP5 | cytoskeleton associated protein 5 |
| C9orf100 | chromosome 9 open reading frame 100 |
| SKA3 | chromosome 13 open reading frame 3 |
| CDC25A | cell division cycle 25 homolog A (S. pombe) |
| ERI2 | exoribonuclease 2 |
| CLSPN | claspin homolog (Xenopus laevis) |
| WDR67 | WD repeat domain 67 |
| HMGB2 | high-mobility group box 2 |
| CDC7 | cell division cycle 7 homolog (S. cerevisiae) |
| SPC24 | SPC24, NDC80 kinetochore complex component, |
| | homolog (S. cerevisiae) |
| UHRF1 | ubiquitin-like with PHD and ring finger domains 1 |
| C12orf48 | chromosome 12 open reading frame 48 |
| MKI67 | antigen identified by monoclonal antibody Ki-67 |
| RPS20 | ribosomal protein S20 |
| C20orf72 | chromosome 20 open reading frame 72 |
| SLBP | stem-loop binding protein |
| CEP55 | centrosomal protein 55 kDa |
| TRIP13 | thyroid hormone receptor interactor 13 |
| AP4B1 | adaptor-related protein complex 4, beta 1 |
| | subunit |
| RRM1 | ribonucleotide reductase M1 |
| DSN1 | DSN1, MIND kinetochore complex component, |
| | homolog (S. cerevisiae) |
| PLK1 | polo-like kinase 1 (Drosophila) |
| DSCC1 | defective in sister chromatid cohesion 1 homolog |
| | (S. cerevisiae) |
| ASPM | asp (abnormal spindle) homolog, microcephaly |
| | associated (Drosophila) |
| FANCA | Fanconi anemia, complementation group A |
| HNRNPUL1 | heterogeneous nuclear ribonucleoprotein U-like 1 |
| STIL | SCL/TAL1 interrupting locus |
| BUB1 | budding uninhibited by benzimidazoles 1 homolog |
| | (yeast) |
| CDCA4 | cell division cycle associated 4 |
| RPRD1B | regulation of nuclear pre-mRNA domain containing |
| | 1B |
| ALG8 | asparagine-linked glycosylation 8, alpha-1,3- |
| | glucosyltransferase homolog (S. cerevisiae) |
| WEE1 | WEE1 homolog (S. pombe) |
| CC2D1A | coiled-coil and C2 domain containing 1A |
| ZWINT | ZW10 interactor |
| TTF2 | transcription termination factor, RNA |
| | polymerase II |
| HAUS8 | HAUS augmin-like complex, subunit 8 |
| STAG1 | stromal antigen 1 |
| KIAA1143 | KIAA1143 |
| BIRC5 | baculoviral IAP repeat-containing 5 |
| CIT | citron (rho-interacting, serine/threonine |
| | kinase 21) |
| CDK1 | cell division cycle 2, G1 to S and G2 to M |
| C12orf32 | chromosome 12 open reading frame 32 |
| FAM200B | hypothetical protein LOC285550 |
| PCNT | pericentrin |
| AFMID | arylformamidase |
| C19orf48 | chromosome 19 open reading frame 48 |
| PSMC3IP | PSMC3 interacting protein |
| CDCA5 | cell division cycle associated 5 |
| ESCO2 | establishment of cohesion 1 homolog 2 |
| | (S. cerevisiae) |
| TMEM111 | transmembrane protein 111 |
| ZFYVE20 | zinc finger, FYVE domain containing 20 |
| CKS1B | CDC28 protein kinase regulatory subunit 1B |
| RANBP1 | similar to RAN binding protein 1; RAN binding |
| | protein 1 |
| MAD2L1 | MAD2 mitotic arrest deficient-like 1 (yeast) |
| ASF1B | ASF1 anti-silencing function 1 homolog B |
| | (S. cerevisiae) |
| INCENP | inner centromere protein antigens 135/155 kDa |
| NUMA1 | nuclear mitotic apparatus protein 1 |
| NOLC1 | nucleolar and coiled-body phosphoprotein 1 |
| UNG | uracil-DNA glycosylase |
| DCAF16 | chromosome 4 open reading frame 30 |
| GEN1 | Gen homolog 1, endonuclease (Drosophila) |
| TROAP | trophinin associated protein (tastin) |
| HNRNPAB | heterogeneous nuclear ribonucleoprotein A/B |
| ATAD5 | ATPase family, AAA domain containing 5 |
| PAQR4 | progestin and adipoQ receptor family member IV |
| DNA2 | DNA replication helicase 2 homolog (yeast) |
| RAB8A | RAB8A, member RAS oncogene family |
| TRIM37 | tripartite motif-containing 37 |
| PBK | PDZ binding kinase |
| CTCF | CCCTC-binding factor (zinc finger protein) |
| TIMELESS | timeless homolog (Drosophila) |
| APITD1 | cortistatin; apoptosis-inducing, TAF9-like |
| | domain 1 |
| TK1 | thymidine kinase 1, soluble |
| INTS7 | integrator complex subunit 7 |
| C15orf42 | chromosome 15 open reading frame 42 |
| MYO9B | myosin IXB |
| BRD9 | bromodomain containing 9 |
| C16orf61 | chromosome 16 open reading frame 61 |
| RFC2 | replication factor C (activator 1) 2, 40 kDa |
| MFSD11 | major facilitator superfamily domain |
| | containing 11 |
| RRM2 | ribonucleotide reductase M2 polypeptide |
| RECQL4 | RecQ protein-like 4 |
| BUB1B | budding uninhibited by benzimidazoles 1 homolog |
| | beta (yeast) |
| PRC1 | protein regulator of cytokinesis 1 |
| E2F2 | E2F transcription factor 2 |
| TRMT2A | TRM2 tRNA methyltransferase 2 homolog A |
| | (S. cerevisiae) |
| CDCA2 | cell division cycle associated 2 |
| DEPDC1B | DEP domain containing 1B |
| SNX5 | sorting nexin 5 |
| NUF2 | NUF2, NDC80 kinetochore complex component, |
| | homolog (S. cerevisiae) |
| XRCC2 | X-ray repair complementing defective repair in |
| | Chinese hamster cells 2 |
| C14orf80 | chromosome 14 open reading frame 80 |
| SHCBP1 | SHC SH2-domain binding protein 1 |
| CEP57 | centrosomal protein 57 kDa |
| KIF20A | kinesin family member 20A |
| DUT | deoxyuridine triphosphatase |
| DNAJC9 | DnaJ (Hsp40) homolog, subfamily C, member 9 |
| NEK2 | NIMA (never in mitosis gene a)-related kinase 2 |
| KIF2C | kinesin family member 2C |
| CEP152 | centrosomal protein 152 kDa |
| KIAA0101 | KIAA0101 |
| CKAP2L | cytoskeleton associated protein 2-like |
| CDCA7 | cell division cycle associated 7 |
| PRKDC | similar to protein kinase, DNA-activated, |
| | catalytic polypeptide; protein kinase, DNA- |
| | activated, catalytic polypeptide |
| ANKRD32 | ankyrin repeat domain 32 |
| KIF15 | kinesin family member 15 |
| UBE2T | ubiquitin-conjugating enzyme E2T (putative) |
| RFC4 | replication factor C (activator 1) 4, 37 kDa |
| FOXM1 | forkhead box M1 |
| FAM54A | family with sequence similarity 54, member A |
| FANCD2 | Fanconi anemia, complementation group D2 |
| C21orf58 | chromosome 21 open reading frame 58 |
| ZNF367 | zinc finger protein 367 |
| SPAG5 | sperm associated antigen 5 |
| VPS29 | vacuolar protein sorting 29 homolog |
| | (S. cerevisiae) |
| AURKB | aurora kinase B |
| CDKN2C | cyclin-dependent kinase inhibitor 2C (p18, |
| | inhibits CDK4) |
| NEIL3 | nei endonuclease VIII-like 3 (E. coli) |
| NUSAP1 | nucleolar and spindle associated protein 1 |
| CDC25C | cell division cycle 25 homolog C (S. pombe) |
| SGOL1 | shugoshin-like 1 (S. pombe) |
| PPWD1 | peptidylprolyl isomerase domain and WD repeat |
| | containing 1 |
| SKA1 | chromosome 18 open reading frame 24 |
| MCM4 | minichromosome maintenance complex component 4 |
| LOC81691 | exonuclease NEF-sp |
| LMNB1 | lamin B1 |
| RBL1 | retinoblastoma-like 1 (p107) |
| C19orf40 | chromosome 19 open reading frame 40 |
| HIST1H3B | histone cluster 1, H3j; histone cluster 1, H3i; |
| | histone cluster 1, H3h; histone cluster 1, H3g; |
| | histone cluster 1, H3f; histone cluster 1, H3e; |
| | histone cluster 1, H3d; histone cluster 1, H3c; |
| | histone cluster 1, H3b; histone cluster 1, H3a; |
| | histone cluster 1, H2ad; histone cluster 2, H3a; |
| | histone cluster 2, H3c; histone cluster 2, H3d |
| CDKN2D | cyclin-dependent kinase inhibitor 2D (p19, |
| | inhibits CDK4) |
| MSH6 | mutS homolog 6 (E. coli) |
| POLD1 | polymerase (DNA directed), delta 1, catalytic |
| | subunit 125 kDa |
| CENPO | centromere protein O |

Gene expression analysis includes measuring the expression of one or more genes of the E2F4 signature in a test sample from a subject. In certain embodiments, at least two, three, four, five, six, seven, eight, nine, ten, twenty, thirty or all of the genes listed in Table 1 are analyzed in accordance with the method of this invention. In particular embodiments, at least two, three, four, five, six, seven, eight, nine, ten, twenty, thirty or all of the genes listed in Table 6 or Table 7 are analyzed in accordance with the method of this invention.

Samples of use in the methods of this invention include a body fluid such as saliva, lymph, blood or urine, or, in particular embodiments, a tissue sample such as a transurethral resection of a bladder tumor or a breast cancer tissue sample. Optimally, there is a sufficient amount of a test sample to obtain a large enough genetic sample to accurately and reliably determine the expression levels of one or more genes of interest. In certain embodiments, multiple samples can be taken from the same tissue in order to obtain a representative sampling of the tissue. A genetic sample can be obtained from the test sample using any techniques known in the art. See, e.g., Ausubel et al. (1999) Current Protocols in Molecular Biology (John Wiley & Sons, Inc., New York); Molecular Cloning: A Laboratory Manual (1989) 2nd Ed., ed. by Sambrook, Fritsch, and Maniatis (Cold Spring Harbor Laboratory Press); Nucleic Acid Hybridization (1984) B. D. Hames & S. J. Higgins eds. The nucleic acid can be purified from whole cells using DNA or RNA purification techniques. The genetic sample can also be amplified using PCR or in vivo techniques requiring subcloning.

Once a genetic sample has been obtained, it can be analyzed for the presence, absence, or level of expression of one or more genes of the E2F4 signature. The analysis can be performed using any techniques known in the art including, but not limited to, sequencing (e.g., serial analysis of gene expression or SAGE), PCR, RT-PCR, quantitative PCR, hybridization techniques, northern blot analysis, microarray technology, DNA microarray technology, Nanostring, flow cytometry, etc. In determining the expression level of a gene or genes in a genetic sample, the level of expression can be normalized as described in the Examples or by comparison to the expression of another gene such as a well-known, well-characterized gene or a housekeeping gene.

In particular embodiments, expression of a gene of interest is determined using microarray technology. Generally, an array is a solid support with peptide or nucleic acid probes attached to the support. Arrays typically include a plurality of different nucleic acid or peptide probes that are coupled to a surface of a substrate in different, known locations. These arrays, also described as microarrays or colloquially “chips” have been generally described in the art, for example, U.S. Pat. Nos. 5,143,854, 5,445,934, 5,744,305, 5,677,195, 6,040,193, 5,424,186 and Fodor, et al. (1991) Science 251:767-777. These arrays may generally be produced using mechanical synthesis methods or light directed synthesis methods which incorporate a combination of photolithographic methods and solid phase synthesis methods. Techniques for the synthesis of these arrays using mechanical synthesis methods are described in, e.g., U.S. Pat. Nos. 5,384,261 and 6,040,193. Although a planar array surface is preferred, the array can be fabricated on a surface of virtually any shape or even a multiplicity of surfaces. Arrays can be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays can be packaged in such a manner as to allow for diagnostics or other manipulation of in an all inclusive device, see for example, U.S. Pat. Nos. 5,856,174 and 5,922,591. The use and analysis of arrays is routinely practiced in the art and any conventional scanner and software can be employed.

The expression data from a particular gene or group of genes can be analyzed using statistical methods described in the Examples to classify, stratify or determine the clinical endpoints of cancer patients. In certain embodiments, changes in transcription factor E2F4 activity in a sample is determined or inferred from the expression data of the one or more genes listed in Table 1, Table 6 or Table 7. In particular, differences in the expression level of E2F4 target genes are used to calculate the activity level of E2F4, wherein increases in E2F4 activity, as compared to a reference, are correlated with a worse survival prognosis in breast cancer, in particular in patients expressing the ER, as well as an increase in breast cancer recurrence or relapse. Increases in E2F4 activity are also correlated with significantly shorter progression-free survival times in bladder cancer patients and as a predictive marker for determining whether IVT should be applied to a NMIBC patient.

Inferred transcription factor activity refers to the quantification of transcription factor activity in a patient sample, which is inferred from information about the transcription factor and transcription factor target gene expression. The activity level of E2F4 can be inferred or calculated using known models including, but not limited to, REACTIN (REgulatory ACTivity Inference; Zhu, et al. (2013) BMC Genomics 14:504), or BASE (Binding Association with Sorted Expression; Cheng, et al. (2007) BMC Bioinformatics 8:452), state-space model (SSM; Li, et al. (2006) Bioinformatics 22:747-54). See also, Wang, et al. (2002) Proc. Natl. Acad. Sci. USA 99:16893). In general, these models generate an activity score for a given transcription factor and sample, wherein, e.g., a score of greater than 0 indicates that the transcription factor activity is increased in the sample and a score of less than 0 indicates that the transcription factor activity is decreased in the sample.

Once transcription factor E2F4 activity in the sample has been inferred, said activity is compared to E2F4 activity in a reference or control. A reference or control can be a sample taken from the same patient, e.g., clinically uninvolved tissue, or can be a sample from one or more healthy subjects. In addition, a reference or control can be the average E2F4 activity from a cohort of healthy individuals.

For the purposes of the present methods, altered E2F4 activity as compared to E2F4 activity in a control or reference sample is indicative of cancer classification, risk of cancer recurrence or relapse, and/or survival. In addition to these identified uses, the analyzed data can also be used to select/profile patients for a particular treatment protocol.

In certain embodiments, the method of the invention permits patients having been determined to have an ER+breast cancer to be classified as belonging to one of two groups, one of these groups being a first group comprising the good prognosis group, and a second group comprising a poor prognosis group, wherein relapse if likely. The good prognosis group may be further defined as comprising ER+ patients with relatively low E2F4 activity. The poor prognosis group may be further defined as comprising ER+ patients with relatively high E2F4 activity. The good prognosis group may be further defined as a group unlikely to benefit from cancer treatment such as chemotherapy or radiation, for example. The poor prognosis group may be further defined as a group likely to benefit from further cancer treatment such as surgery, chemotherapy and/or radiation therapy, for example.

According to a further embodiment, when a NMIBC patient demonstrates a relatively low E2F4 activity, this identifies the patient as being unlikely to benefit from intravesical BCG immunotherapy, whereas a patient demonstrating a relatively high E2F4 activity identifies that patient as receiving a likely benefit from intravesical BCG immunotherapy.

In certain aspects of the invention, the methods employ a computer to analyze expression data, calculate E2F4 activity and carry out comparisons with a reference. For example, in one embodiment, a computer running a software program analyzes gene expression level data from a patient, runs one or models to assign an E2F4 score to a sample, compares that score to a reference score or distribution of scores from a population of patients having the same disease state, and determines the prognosis for the patient as being good or poor. For example, the software is capable of generating a report summarizing the patient's gene expression levels and/or the patient's E2F4 scores, and/or a prediction of the likelihood of long-term survival of the patient and/or the likelihood of recurrence or relapse of the patient's disease condition, i.e., cancer. Further, in one embodiment, the computer program is capable of performing any statistical analysis of the patient's data or a population of patient's data as described herein in order to generate an E2F4 score for the patient. Further, in one embodiment, the computer program is also capable of normalizing the patient's gene expression levels in view of a standard or control prior to inferring E2F4 activity. Further, in one embodiment, the computer is capable of ascertaining raw data of a patient's expression values from, for example, immunohistochemical staining or a microarray, or, in another embodiment, the raw data is input into the computer.

The following non-limiting examples are provided to further illustrate the present invention.

### EXAMPLE 1

### Materials and Methods

Collection of Gene Expression Cell Cycle Data. Human cell cycle gene expression profiles collected in HeLa S3 cells using two-channel cDNA arrays (Whitfield, et al. (2002) Mol. Biol. Cell 13:1977-2000) were downloaded from the NCBI Gene Expression Omnibus (GEO, (Barrett & Edgar (2006) Method Enzymol. 411:352-69; GSE3497). The dataset contained expression profiles from five independent time courses, wherein the course with the largest number of time points (N=48) was used for this analysis.

Collection of Gene Expression and Breast Cancer Patient Clinical and Survival Data. Using collated meta-analysis as a guide (Ur-Rehman, et al. (2013) Breast Cancer Res. Treat. 139:907-21), the ROCK, GEO, and NIH PUBMED databases were queried to access and download all publically available breast cancer gene expression datasets for which standard clinical data (age at diagnosis, estrogen receptor status, tumor size, grade, and lymph node involvement) and survival outcome data (either distant metastasis free survival “dmfs” or relapse free survival “rfs”) were present for a minimum of 150 samples. This resulted in the collection of 1902 unique breast cancer samples across eight different datasets and on both one and two-channel arrays (Table 2).

TABLE 2

| GSE ID | Platform | # Samples | Source |
|---|---|---|---|
| — | cDNA two | 295 | van der Vijver, et al. (2002) |
| | channel | | N. Engl. J. Med. 347: 1999-2009 |
| GSE1456 | HG-U133A | 159 | Pawitan, et al. (2005) Breast |
| | | | Cancer Res. 7: R953-64 |
| GSE2034 | HG-U133A | 286 | Wang, et al. (2005) Lancet |
| | | | 365: 671-9 |
| GSE2990 | HG-U133A | 177 | Sotiriou, et al. (2006) J. |
| | | | Natl. Cancer Inst. 98: 262-72 |
| GSE3494 | HG-U133A | 260 | Miller, et al. (2005) PNAS |
| | | | 102 : 13550-5 |
| GSE6532 | HG-U133A | 327 | Loi, et al. (2008) BMC |
| | | | Genomics 9: 239 |
| GSE7390 | HG-U133A | 198 | Desmedt, et al. (2007) Clin. |
| | | | Cancer Res. 13: 3207-14 |
| GSE11121 | HG-U133A | 200 | Schmidt, et al. (2008) Cancer |
| | | | Res. 68: 5405-13 |

For each sample, composite predictive measures derived from clinical data, the Nottingham Prognostic Index (NPI) and Adjuvant!Online scores, were calculated and recorded. The Adjuvant! risk score of “high” or “low” was derived from the Adjuvant!Online numerical scores following established procedures (Loi, et al. (2008) BMC Genomics 9:239), while the NPI risk scores of “low,” “medium,” or “high” were derived from the standard numerical score ranges of <3.4 , 3.4-5.4, and >5.4, respectively.

Definition of the E2F4 Target Gene Signature. All publically-available E2F4 ChIP-Seq datasets were accessed and downloaded, resulting in the collection of E2F4 chromatin binding data in the GM06900, HeLa, and K562 cell lines (Lee, et al. (2011) Nucl. Acids Res. 39:3558-73; Desmedt, et al. (2007) Clin. Cancer Res. 13:3207-14). With a threshold False Discovery Rate of 1%, the TIP probabilistic method (Schmidt, et al. (2008) Cancer Res. 68:5405-13) was used to determine the candidate target genes of E2F4 in each cell line, resulting in the identification of 428, 438, and 429 target genes in the GM06990, HeLa, K562 and cells lines, respectively. The 199 identified target genes (Table 1) shared across the three cell lines were selected as the E2F4 target gene signature.

Calculation of iRASs for E2F4 in Cancer Samples. The REACTIN algorithm, as introduced and previously described (Zhu, et al. (2013) BMC Genomics 14:504), was applied to all collected cancer samples using the E2F4 target gene signature and with a minimum of 10,000 permutations. Briefly, REACTIN sorts the relative expression levels of all genes in a given sample and generates two cumulative distribution functions to summarize the expression levels of a target gene set and non-target gene set of a chosen TF—here, E2F4. REACTIN then uses the differential scores, calculated by comparing the two functions, to obtain the individual regulatory activity score (iRAS) for E2F4 in each tumor sample. These resulting iRASs are scores similar to the values of the D-statistic in the KS-test (Kolmogorov-Simonov test) and reflect the regulatory activity of E2F4 in a sample, with a higher iRAS value indicating a higher E2F4 regulatory activity as compared to a lower iRAS value.

For gene expression data measured by two-channel arrays, the expression levels of genes are represented as relative values: the log ratios of genes in a sample with respect to a control. In this case, the expression data can be directly used as input to the REACTIN method. However, for gene expression data from one-channel arrays, the absolute expression levels of genes are provided, which cannot be directly taken as input. To manage this problem, gene-wise median normalization was performed to convert the data into relative expression values. Specifically, median expression level for each gene across all samples was calculated and this median was subtracted from all values. This median normalization was performed in log-transformed absolute expression values, thus making post-normalization data somewhat similar to the log ratios captured by two-channel arrays.

Survival Analyses. Cox PH models were used to examine if E2F4 activity correlated with patient survival outcomes. Both univariate and multivariate regression models with E2F4 iRASs alone, or E2F4 iRASs plus confounding variables (ER status, tumor stage, grade, etc.), respectively, were investigated. Where indicated, E2F4 iRASs were dichotomized into positive score and negative score groups, enabling E2F4 iRASs to be treated as a binary variable throughout the analyses. Kaplan-Meier survival curves derived from the Cox PH models were also generated. For the breast cancer samples, analyses were performed both within each individual dataset and across the aggregated dataset derived from all individual datasets pooled together, as indicated. Analyses were performed in R using the “survival” package, specifically using the “survreg” and “coxph” functions to construct the Cox PH models and the “survdiff” function to compare the difference between two survival curves.

Determination of Intrinsic Subtypes of Breast Cancer Samples. Breast cancer samples were classified into the five intrinsic subtypes, Basal-like, Luminal A, Luminal B, Her-2 enriched, and Normal-like (Kim, et al. (2010) Mol. Cancer 9:3), using the PAM50 algorithm (Lee, et al. (2008) BMC Med. Genomics 1:52) after having their gene expression values median-centered as recommended (Lee, et al. (2008) Clin. Cancer Res. 14:7397-404). Namely, Spearman correlation coefficients between the median-centered expression values in each sample and the provided PAM50 centroids for each of the five intrinsic subtypes were calculated. Samples were assigned to the subtype for which they had the highest Spearman correlation coefficient. Samples with correlations less than 0.1 for all subtypes were excluded from subsequent analysis.

Oncotype DX Analysis. The Recurrence Scores of breast cancer samples (ER positive, lympo node negative) were calculated using a 21-gene signature proposed by Oncotype DX (Smith, et al. (2010) Gastroenterology 138:958-68). Based on the scores, samples were stratified into Low, Intermediate and High Risk groups. The R package “genefu” was used to implement the Oncotype DX analysis.

Collection of Gene Expression and Additional Cancer Patient Data. In addition to breast cancer data, data was collected for six other cancer types, including bladder cancer, glioblastoma, non-small cell lung cancer, colon cancer, acute myeloid leukemia and Burkitt's lymphoma (Table 3).

TABLE 3

| GSE ID | Platform | Cancer Type | # Samples | Source |
|---|---|---|---|---|
| GSE13507 | Illumina | Bladder | 256 | Kim, et al. |
| | beadchip | | | (2010) Mol. |
| | | | | Cancer 9: 3 |
| GSE1827 | | Bladder | 80 | |
| GSE19915 | | Bladder | 160 | Lindgren, et |
| | | | | al. (2010) |
| | | | | Cancer Res. |
| | | | | 70: 3463-72 |
| GSE31684 | | Bladder | 93 | Riester, et al. |
| | | | | (2012) Clin. |
| | | | | Cancer Res. |
| | | | | 18: 1323-33 |
| GSE32894 | | Bladder | 308 | Sjodahl, et al. |
| | | | | (2012) Clin. |
| | | | | Cancer Res. |
| | | | | 18: 3377-86 |
| GSE13041 | HG-U133A | Glioblastoma | 191 | Lee, et al. |
| | | | | (2008) BMC Med. |
| | | | | Genomics 1: 52 |
| GSE8894 | HG-U133A | Non-small | 138 | Lee, et al. |
| | Plus 2 | cell lung | | (2008) Clin. |
| | | | | Cancer Res. |
| | | | | 14: 7397-7404 |
| GSE17536 | HG-U133A | Colon | 177 | Smith, et al. |
| | Plus 2 | | | (2010) |
| | | | | Gastroenterol. |
| | | | | 138: 958-68 |
| GSE425 | cDNA two | Acute | 119 | Bullinger, et |
| | channel | myeloid | | al. (2004) NEJM |
| | | leukemia | | 350: 1605-16 |
| GSE4475 | HG-U133A | Burkitt's | 221 | Hummel, et al. |
| | | lymphoma | | (2006) NEJM |
| | | | | 354: 2419-30 |

In the GSE13507 dataset, 10, 58, 165 and 23 samples were from normal bladder tissues, normal bladder tissue surrounding bladder tumors, primary bladder tumors, and recurrent bladder tumors, respectively. Probeset expression was converted into gene expression for all datasets. For genes with multiple probesets, the one with the highest average intensity in all samples was selected to represent the corresponding genes.

The ChIP-seq datasets for E2F4 were downloaded as wig files from previous publications, providing genome-wide occupation of E2F4 in GM06900 (Lee, et al. (2011) Nucl. Acids Res. 39:3558-73), HeLa, and K562 (Gerstein, et al. (2012) Nature 489:91-100) cell lines. The probabilistic method TIP (Target Identification from Profiles) (Cheng, et al. (2011) Bioinformatics 27:3221-7) was used to identify E2F4 target genes in each cell line using a threshold of FDR<0.01 (False Discovery Rate). Genes shared in the three cell lines were then identified, resulting in an E2F4 core gene set with 199 genes.

Preparation of Meta-Bladder Datasets. Two meta-bladder cancer datasets were generated, which contained samples with matched gene expression profiles and survival information. The first meta-dataset included a total of 482 primary bladder tumor samples from three one-channel datasets, GSE13507, GSE31684 and GSE32894 (Kim, et al. (2010) Mol. Cancer 9:3; Sjodahl, eta 1. (2012) Clin. Cancer Res. 18:3377-86; Riester, et al. (2012) Clin. Cancer Res. 18:1323-33). All of the samples were renormalized by quantile normalization to have the same distribution at the gene level (Bolstad, et al. (2003) Bioinformatics 19:185-93). Then expression values were log transformed and gene-wise median normalization was performed to convert the data into relative expression values. After median normalization, the median expression values in the 482 samples for all genes were zeros. The second meta-dataset included a total 240 primary bladder tumor samples from two two-channel arrays, GSE1827 and GSE19915 (Lindgren, et al. (2010) Cancer Res. 70:3463-72). The dataset contained the relative expression values (log ratios) of genes against a reference sample (RNA pooled from 10 human cell lines). No additional processing was performed for this meta-dataset.

Calculation of E2F4 Scores in Bladder Cancer. Given a bladder cancer dataset or a meta-dataset, an algorithm called BASE (Binding Association with Sorted Expression) was applied to infer E2F4 activity in all of the samples (Cheng, et al. (2007) BMC Bioinformatics 8:452). The BASE algorithm sorts genes based on their relative expression levels in a sample, and then summarizes the distribution of the E2F4 target genes in the ranked gene list using a non-linear random walk-based method. For each sample, BASE gives rise to an E2F4 score. A positive E2F4 score indicates that E2F4 targets tend to be highly expressed in the ranked gene list, implying high E2F4 activity in the sample. Conversely, a negative E2F4 score indicates that E2F4 targets tend to be lowly expressed in the ranked gene list, and therefore implying low E2F4 activity in the sample. In general, the E2F4 scores follow a bimodal distribution with two peaks on the positive and negative sides, respectively.

Statistical Analysis. To investigate the effectiveness of E2F4 program for predicting prognosis, bladder cancer samples were dichotomized into E2F4>0 and E2F4<0 groups. Kaplan-Meier survival curves were derived from the Cox proportional hazard models (Cox (1972) J. Royal Stat. Soc., Series B 34:187-220). The difference between the survival curves of the two groups was compared with significance being estimated by using log-rank test. Analyses were performed in R using the “survival” package. Specifically the “survfit” function was called to create Kaplan-Meier survival curves, and the “survdiff” function was called to compare the difference between two survival curves.

### EXAMPLE 2

### E2F4 Regulatory Program Predicts Patient Survival Prognosis in Breast Cancer

The E2F4 Target Gene Signature Contains Cell Cycle Regulators and is Enriched for Genes that Correlate with Patient Survival. Leveraging E2F4 ChIP-Seq data from experiments performed across HeLa and K562 (Desmedt, et al. (2007) Clin. Cancer Res. 13:3207-14) and GM06990 (Lee, et al. (2011) Nucl. Acids Res. 39:3558-73) cell lines, the TIP method (Schmidt, et al. (2008) Cancer Res. 68:5405-13) was used to identify E2F4 target genes in each cell line at a P-value<0.01 confidence level. In HeLa, K562 and GM06990 cell lines, 438, 429, and 428 target genes, respectively, were identified, of which 199 were found to overlap across the three cell lines. This shared group was defined as the E2F4 target gene signature. Examination of this gene signature using DAVID Functional Annotation Clustering against a Homo sapiens gene background produced 58 clusters related to cell cycle regulation, mitosis, and microtubule organization; kinetochore; DNA repair; DNA replication; nucleoplasm; meiotic cell cycle, and nucleotide binding. This confirmed the significance of this gene signature to cell cycle, matching the known important role played by E2F4 in cell cycle arrest and/or progression (Schwemmle & Pfeifer (2000) Int. J. Cancer 86:672-77; Lee, et al. (2011) Nucl. Acids Res. 39:3558-73).

To examine how these 199 E2F4 target genes might relate to survival, the correlation of their expression was compared with survival to that of all genes in an initial dataset (van de Vijver, et al. (2002) N. Engl. J. Med. 347:1999-2009). Cox regression analysis was carried out for each gene and 751 of them were found to be significantly correlated with patient survival times (disease-free survival time, dfs). Of these genes, 58 were E2F4 targets with an enrichment of 8-fold (P=8e-40, Fisher's exact test). After taking confounding factors such as ER status and positive lymph node involvement into account in the model, 83 significant genes were identified, 17 of which were E2F4 targets with an enrichment of 21-fold (P=2e-18, Fisher's exact test). These results indicate that the selected E2F4 signature genes are enriched for genes with predictive ability for patient survival in breast cancer.

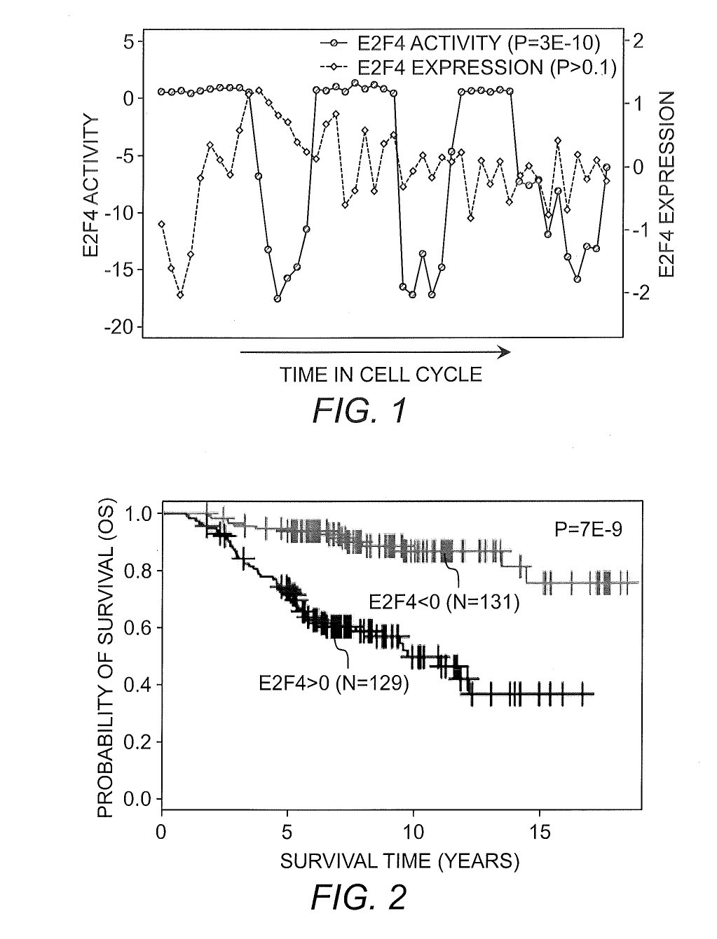

E2F4 iRASs Outperform E2F4 Expression Levels as Markers of Cell Cycle Phase. To test the E2F4 target gene signature as an indicator of E2F4′s regulatory activity, regulatory activity was compared to E2F4′s mRNA expression level and how it correlates to cell cycle phase in a HeLa S3 cell cycle dataset (Whitfield, et al. (2002) Mol. Biol. Cell 13:1977-2000). As E2F4 is a known critical cell cycle regulator, its activity cycles with cell cycle phase. Using REACTIN and E2F4′s target gene signature, the iRASs of E2F4 was calculated throughout the cell cycle. These iRASs showed a significant periodical pattern (P=3e-10, Fisher's G test), while the expression levels of E2F4 do not (P>0.1, Fisher's G test) (FIG. 1). It was concluded that REACTIN-derived E2F4 RASs more accurately reflected E2F4 regulatory activity than did E2F4 expression levels.

E2F4 iRASs Predict Breast Cancer Survival Prognosis. It has been shown that E2F4 activity inferred from expression of all genes predicts patient survival prognosis of breast cancer patients (Zhu, et al. (2013) BMC Genomics 14:504). For each breast cancer sample of the Vijver dataset (van de Vijver, et al. (2002) N. Engl. J. Med. 347:1999-2009), an E2F4 iRAS was generated using REACTIN based on the sorted relative expression levels of the E2F4 target genes in the sample. The survival prediction with these iRASs scores was compared to survival prediction with two commonly considered pathological variables in breast cancer therapy: lymph node status (whether the cancer has metastasized to the nodes or not), and estrogen receptor (ER) status, i.e., whether the tumor overexpresses the ER, which would suggest that its growth is driven by estrogen and is consequently responsive to hormonal therapy targeting the ER's signal transduction function (Bullinger, et al. (2004) N. Engl. J. Med. 350:1605-16; Hummel, et al. (2006) N. Engl. J. Med. 354:2419-30). Looking at patient outcome data, a Cox PH model showed that E2F4 iRASs improved survival prediction over ER and lymph node status alone (Table 4).

TABLE 4

| | | Coef- | | | | |
|---|---|---|---|---|---|---|
| | | ficient | Std. | P- | Hazard | |
| Variable | Type | (β) | error | Value | ratio | 95% CI |
| E2F4 | Continuous | 0.102 | 0.023 | 1.19E−05 | 1.11 | 1.05-1.16 |
| Score | | | | | | |
| ER | Binary | −0.594 | 0.269 | 0.027 | 0.55 | 0.33-0.94 |
| Status | | | | | | |
| Pos. | Binary | −0.005 | 0.255 | 0.98 | — | — |
| node | | | | | | |

After dichotomizing E2F4 iRASs into two groups of high activity, E2F4 iRAS>0 and low activity, E2F4 iRAS<0, a Kaplan-Meier plot comparing the two groups recapitulates this finding (FIG. 2; significance of difference between curves, P=7e-9), with the E2F4>0 group associated with worse prognosis. In contrast, the expression level of E2F4 itself does not significantly predict survival prognosis (P>0.4), mirroring the FIG. 1 finding that activity scores are a better indicator of E2F4 function than expression levels alone.

To ensure that these results were not limited to the Vijver dataset, all additional publically available breast cancer datasets were obtained for which survival and clinicopathological data were available for at least 150 samples (Table 2). As with the samples in the Vivjer dataset, iRASs were calculated for each sample and were dichotomized into high E2F4 activity (E2F4 iRAS>0) and low E2F4 activity (E2F4 iRAS<0) groups. Kaplan-Meier survival plots were then generated separately for each dataset, using as the survival endpoint whichever variable (overall survival, relapse-free survival, or distant metastasis free survival) was most complete. In all seven of the datasets, E2F4 iRASs significantly predict survival outcome (all P-Values<0.05). As with the Vijver dataset, higher E2F4 activity was predictive of worse survival prognosis.

Moreover, similar analysis was carried out with the breast cancer metadata downloaded from the ROCK database, which provided normalized gene expression profiles and clinical information for 1570 breast cancer samples. The E2F4 iRASs were calculated for all samples and were dichotomized into positive and negative groups. Survival analysis indicated that the relapse-free survival times of the positive groups were significantly shorter than those of the negative groups (P=4e-8). After controlling for many clinical variables including patient age, tumor size, grade, ER status and lymph node status, the E2F4 iRAS was still highly significant in predicting patient relapse-free survival (rfs) times (P=6e-6) in Cox survival regression model.

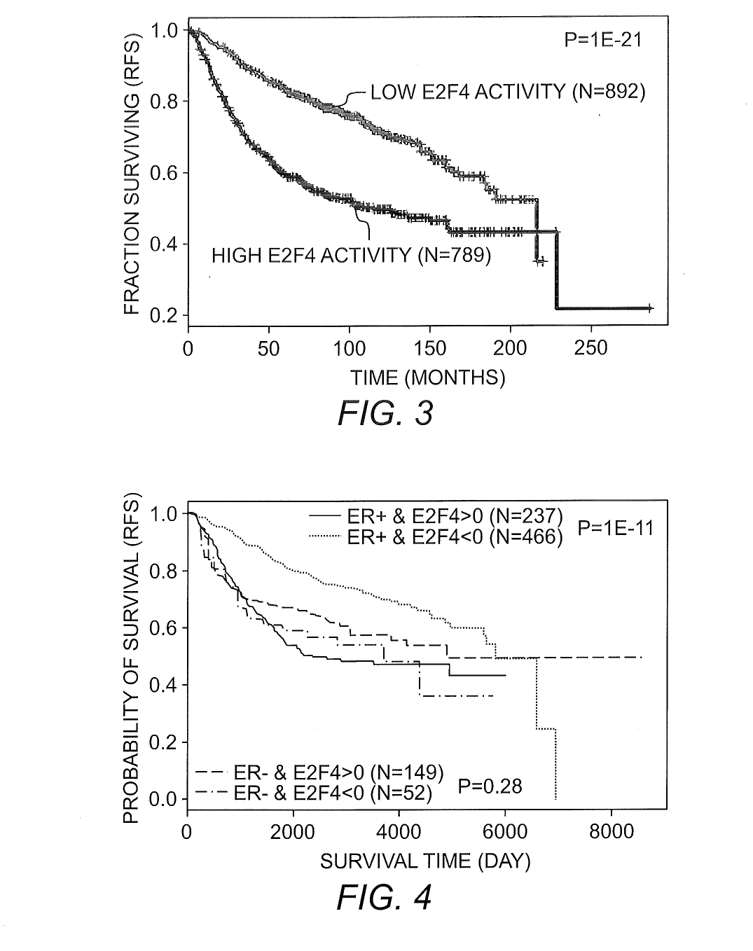

E2F4 iRASs Remain Predictive of Survival Prognosis After Pooling and Adjustment for Clinicopathological Data. Based on the results with individual breast cancer datasets, REACTIN was tested on a larger dataset, as the increased sample size from pooling would enable stratification and adjustment for other variables. Since iRASs are normalized values, it was possible to pool them to conduct aggregate analyses across data points. Combining together the samples from all eight breast cancer datasets, a Kaplan-Meier plot of the pooled data recapitulated the previous findings (FIG. 3, significance of difference between curves, P=le-21). As detailed in Example 1, clinical data (age at diagnosis, estrogen receptor status, tumor size, tumor grade, and lymph node involvement) were collected for all breast cancer samples and used to calculate clinical risk scores using the Nottingham Prognostic Index and Adjuvant!Online formulae. The pharmacological treatment status of each sample, whether chemotherapy and/or hormone therapy, was additionally recorded.

Inclusion of these clinicopathological covariates in Cox PH models of the pooled samples resulted in adjusted E2F4 iRAS Hazard Ratios that were positive and statistically significant (Table 5). Regardless of model chosen (Table 5; Models A, B, and C), E2F4 iRASs significantly predicted survival outcome, with a high E2F4 iRAS resulting in a worse survival prognosis than low E2F4 iRAS data points (HRs>1.00, P-values<0.001 in all cases). Graphically, Kaplan-Meier plots of the pooled data, stratified by pharmacological treatment status and composite clinical risk, exhibited these findings as well. E2F4 iRASs provided additional prognosis prediction beyond the commonly collected clinicopathological variables alone.

TABLE 5

| | | | | | |
|---|---|---|---|---|---|
| | | Hazard | Std. | 95% | |
| Variable | Type | Ratio | Error | CI | P-Value |

A

| | | | | | |
|---|---|---|---|---|---|
| E2f4 iRAS | Binary | 2.013 | 0.108 | 1.63-2.49 | 8.54E−11 |
| (High vs. Low) | | | | | |
| Age | Continuous | 1.002 | 0.004 | 0.99-1.01 | 0.6890 |
| ER Status | Binary | 1.061 | 0.113 | 0.85-1.33 | 0.6029 |
| (+vs.−) | | | | | |
| Grade | Ordinal | 1.157 | 0.074 | 1.01-1.34 | 0.0475 |
| Size | Continuous | 1.013 | 0.004 | 1.01-1.02 | 0.0001 |
| Lymph Node | Binary | 1.407 | 0.149 | 1.05-1.88 | 0.0215 |
| Status | | | | | |
| (+vs.−) | | | | | |
| Pharma- | Binary | 0.651 | 0.148 | 0.49-0.87 | 0.0037 |
| cological | | | | | |
| Treatment | | | | | |

B

| | | | | | |
|---|---|---|---|---|---|
| E2f4 iRAS | Binary | 1.9013 | 0.0918 | 1.59-2.28 | 2.57E−12 |
| (High vs. Low) | | | | | |
| Adjuvant!Risk | Binary | 0.6799 | 0.1001 | 0.56-0.83 | 0.0001 |
| Score | | | | | |
| (Low vs. High) | | | | | |
| Pharma- | Binary | 0.8362 | 0.091 | 0.70-0.99 | 0.0493 |
| cological | | | | | |
| Treatment | | | | | |

C

| | | | | | |
|---|---|---|---|---|---|
| E2f4 iRAS | Binary | 1.86177 | 0.10 | 1.52-2.27 | 1.12E−09 |
| (High vs. Low) | | | | | |
| NPI Score | Continuous | 1.29314 | 0.05844 | 1.15-1.45 | 1.09E−05 |
| Pharma- | Binary | 0.76527 | 0.09927 | 0.63-0.93 | 0.0070 |
| cological | | | | | |
| Treatment | | | | | |

Whether clinicopathological covariates were considered separately (Model A) or combined into either the stratified Adjuvant!Online score (Model B) or the Nottingham Prognostic Index (Model C), E2F4 iRASs significantly predicted survival outcome, with a high E2F4 iRAS resulting in worse survival prognosis (HRs > 1.00, p-values < .001 in all cases). Survival endpoint was relapse-free survival for all three tables. Distant metastasis-free survival and overall survival endpoints recapitulated these results. Results represent the pooled sample data of all eight breast cancer datasets (Table 2). For Model A, n = 1349; Model B, n = 1511; Model C, n = 1369.

E2F4 iRASs Predict Patient Survival Prognosis Within Different Histological Subtypes. As indicated, ER status is a key factor in planning breast cancer therapy. ER status was of interest as a potential confounding factor for analysis after a review of E2F4 and breast cancer literature suggested a link between E2F4/Cyclin E levels and cancer cell proliferation in ER-dependent tumors (Galea, et al. (1992) Breast Cancer Res. Treat. 22:207-219). Therefore, to account for confounding by ER status, positive and negative E2F4 score patient groups were further divided by their ER status (whether the tumors express ER or do not express it) and survival curves were compared. Interestingly, it was observed that E2F4 regulatory activity was significantly correlated with survival only in patients expressing the ER (P=6e-12), and was not significant (P>0.1) in patients who did not express ER (FIG. 4). Furthermore, an examination of E2F4 activity distribution in ER+ versus ER− patients showed significantly lower levels of E2F4 activity in the ER+ group (P=3e-10, Wilcoxon rank sum test). A similar pattern was seen with the progesterone receptor (PR) status, which is usually tested along with the ER status, where E2F4 was significantly correlated with survival in PR+ (P=2e-5) but not PR− patients (P>0.1). This was expected, since tumors that are ER+ tend to be PR+ as well. In contrast to ER and PR status, p53 staining and MYC levels did not prove to be significant confounders of E2F4-DMFS.

E2F4 iRASs Correlate With the Survival Prognosis of Intrinsic Breast Cancer Subtypes. It has become increasingly understood that breast cancers segregate by gene expression into different intrinsic subtypes, with the assumption that cancers falling within the same subtype share a similar prognosis and suggested therapy method. Several breast cancer subtypes have been defined in the art, including luminal A, luminal B, HER2-enriched, basal-like, and normal-like cancers (Lee, et al. (2008) BMC Med. Genomics 1:52). In a pooled analysis of the eight breast cancer datasets, a Kaplan Meier plot of each sample classified into one of these intrinsic subtypes showed that subtypes had different survival prognoses. Consistent with previous reports (Parker, et al. (2009) J. Clin. Oncol. 27:1160-7), the subtypes fell from good to poor prognosis in the order of Luminal A, Normal-like, Basal-like, Luminal B and Her-2 enriched. Furthermore, the prognosis of these different molecular subtypes was strongly correlated with E2F4 iRAS: a high fraction of samples with positive E2F4 iRASs fell into the poor prognostic subtypes (Her-enriched, Luminal B and Basal-like), whereas in good prognostic subtypes (Luminal A and Normal-like), the fraction of samples with a positive E2F4 iRAS was much lower. These results indicated that the survival prognoses of different intrinsic subtypes can be at least partially reflected by the E2F4 regulatory program.

### EXAMPLE 3

### E2F4 Program is Predictive of Progression and Intravesical Immunotherapy Efficacy in Bladder Cancer

Overview of Analysis. Given a gene expression dataset for a number of bladder tumor samples, a method called BASE was used to infer the regulatory activities of E2F4 (denoted as E2F4 scores) in these samples. The E2F4 scores were calculated based on the expression of a core set of E2F4 target genes identified from ChIP-seq experiments. When target genes are highly expressed in a sample, BASE results in a positive E2F4 score, indicating high E2F4 activity in this sample. Conversely, when target genes are lowly expressed, BASE results in a negative E2F4 score, indicating low E2F4 activity in the corresponding sample. The core E2F4 target genes represent a set of genes that are regulated by E2F4 in a non-tissue-specific manner (Table 2). They were identified as the E2F4 targets shared in multiple human cell lines (K562, GM12878 and HeLa) defined from ChIP-seq data.

Bladder tumor samples were then stratified into high-risk (E2F4>0) and low-risk (E2F4<0) groups based on their E2F4 scores. The survival times of the two groups were compared to examine whether E2F4 scores are predictive of bladder cancer prognosis. The E2F4 program was first tested for survival prediction in the GSE13507 dataset that contained expression profiles for normal and tumorous bladder samples (Sanchez-Tillo, et al. (2012) Cell. Mol. Life Sci. 69:3429-56). Different survival times were tested including overall survival time (OS), cancer specific survival time (CSS), recurrence-free survival time (RFS), and progression-free survival time (PFS). Then the findings were validated in two meta-bladder datasets that combined samples from multiple experiments using a one-channel platform and a two-channel platform, respectively.

E2F4 Scores in Different Subsets of Bladder Samples. First, the E2F4 activities were compared in different subsets of samples contained in the GSE13507 dataset. The dataset was composed of 256 samples, including 10 normal bladder samples, 58 normal samples surrounding bladder tumors, 165 primary bladder tumor samples, and 23 recurrent bladder tumor samples. As expected, the E2F4 scores were significantly higher in tumor samples (primary and recurrent) than in normal bladder samples (normal and surrounding) (P=2E-17, Wilcox rank sum test). Of the samples, 53% of primary (88/165) and 73% of recurrent tumor samples (16/23) had positive E2F4 scores, whereas the majority of normal samples had negative E2F4 scores: 86% of surrounding (50/58) and 100% normal bladder samples. Compared to the normal samples, surrounding samples showed slightly higher E2F4 scores (P=0.02, Wilcox rank sum test), indicating these “normal” bladder samples might be contaminated with tumor cells. Compared with primary tumor samples, the recurrent tumor samples showed higher E2F4 scores (P=0.03, Wilcox rank sum test). The primary tumor samples were collected from patients with or without recurrence during follow-up. The primary tumor samples from recurrent patients had a larger fraction of positive E2F4 scores than those from non-recurrent patients (58% versus 36%), but the difference of E2F4 scores between these two groups were not significant (P>0.05, Wilcox rank sum test). Similarly, for the recurrent patients, their primary tumors and recurrent tumors exhibited no significant difference in their E2F4 scores (P>0.05, Wilcox rank sum test).

The primary tumor samples in this dataset were from different stages that included 24 Ta, 80 T1, 31 T2, 19 T3 and 11 T4 samples. The E2F4 scores demonstrated an increasing trend from Ta to T4. When superficial samples (Ta and Ti) and invasive samples (T2-T4) were compared, a significant difference was observed in their E2F4 scores (P=0.0007, Wilcox rank sum test). In addition, when primary tumor samples with different grade were compared, the G2 group showed significantly higher E2F4 scores than the G1 group (P=8E-9, Wilcox rank sum test). Taken together, these results indicate that E2F4 of samples are highly correlated with their clinical factors such as tumor stage, grade and the recurrence of patients.

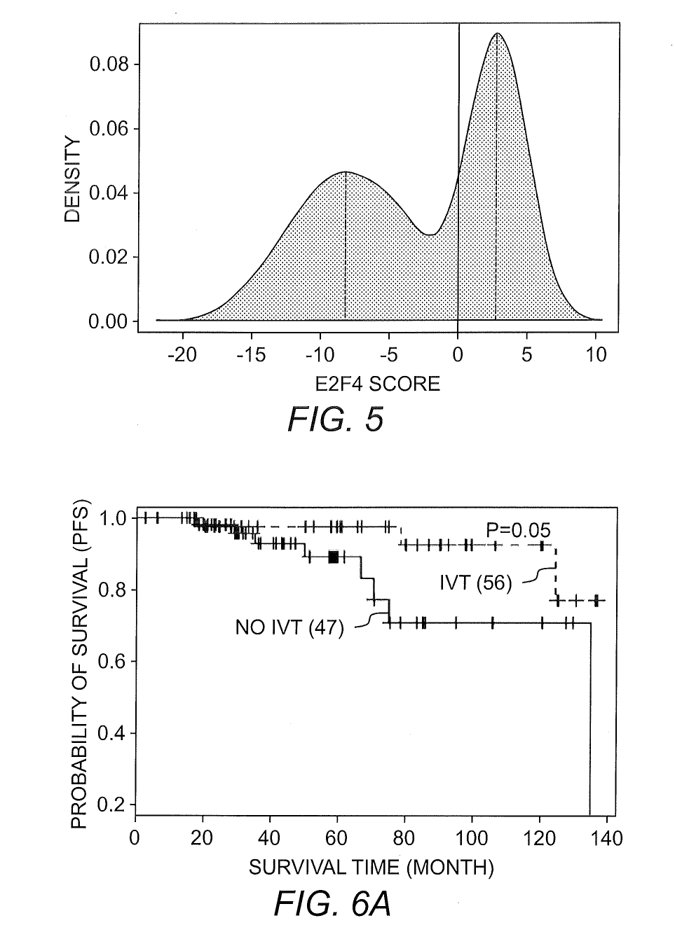

E2F4 Program is Predictive of Survival of Bladder Cancer Patients. The primary bladder tumor samples of the GSE13507 dataset were subsequently analyzed using the E2F4 scores to predict patient survival. Since the survival of patients can be complicated by treatment, samples from patients treated with systemic chemotherapy were excluded, resulting in 138 primary samples. This analysis indicated that E2F4 scores have a bimodal distribution with positive and negative peaks (FIG. 5), which enabled the stratification of patients in two different ways. First, patients were simply divided into positive (E2F4>0) and negative (E2F4<0) groups. The E2F4>0 group showed significantly shorter cancer-specific survival time than the E2F4<0 group (P=0.0008). At the median follow-up time (40 months), 23% of E2F4>0 patients but only 4% E2F4<0 patients die from cancer. Second, the E2F4 scores were determined at the positive and the negative peaks (see dashed lines in FIG. 5) and were used as the cut-off values to divide patients into high-, intermediate- and low-risk groups. This analysis indicated that the three groups showed a significant difference in their cancer-specific survival times.

In additional to cancer-specific survival time, the capacity of the E2F4 program for predicting overall survival, recurrence-free survival and progression-free survival of patients were tested. E2F4 scores were predictive of all these types of survival, with the highest accuracy achieved for progression-free survival of patients. Moreover, the same analyses were repeated using all of the 165 primary tumor samples (i.e., without filtering out systemic chemotherapy treated patients), and similar results were obtained.

Application of E2F4 Program to NMIBC and MIBC. In the 165 primary bladder tumor samples, 103 were NMIBC (non-muscle invasive at Ta or T1 stages, also called superficial tumor) and 52 were MIBC (muscle invasive at T2, T3 or T4 stages). After excluding systemic chemotherapy-treated patients, 102 NMIBC and 36 MIBC samples were obtained. Using these samples, the effectiveness of the E2F4 program for predicting progression-free survival in both subtypes was analyzed. The results indicated that the program was valid in both NMIBC and MIBC. It is known that tumor grade is correlated with patient survival, and it was shown that E2F4 scores were significantly different between G1 and G2 samples. Thus, the E2F4 program was next tested in 93 G1 samples without being treated by systemic chemotherapy. This analysis indicated that E2F4>0 patients showed significantly shorter progression-free survival times than E2F4<0 patients in all G1 samples as well as in the NMIBC G1 samples.

Similar results were identified when all primary tumor samples (with or without systemic chemotherapy) were used for above analysis. However, lower predictive powers of the E2F4 program were observed for IMBC samples. The dataset contained 52 and 36 MIBC samples, respectively, before and after systemic chemotherapy exclusion. When all of the 52 MIBC samples were used, a significant difference in survival between the E2F4>0 and the E2F4<0 groups (P=0.2) was not observed in spite of more samples being included. This indicates that systemic chemotherapy does have an effect on the progression of patients and including treated samples complicates the prognostic analyses.

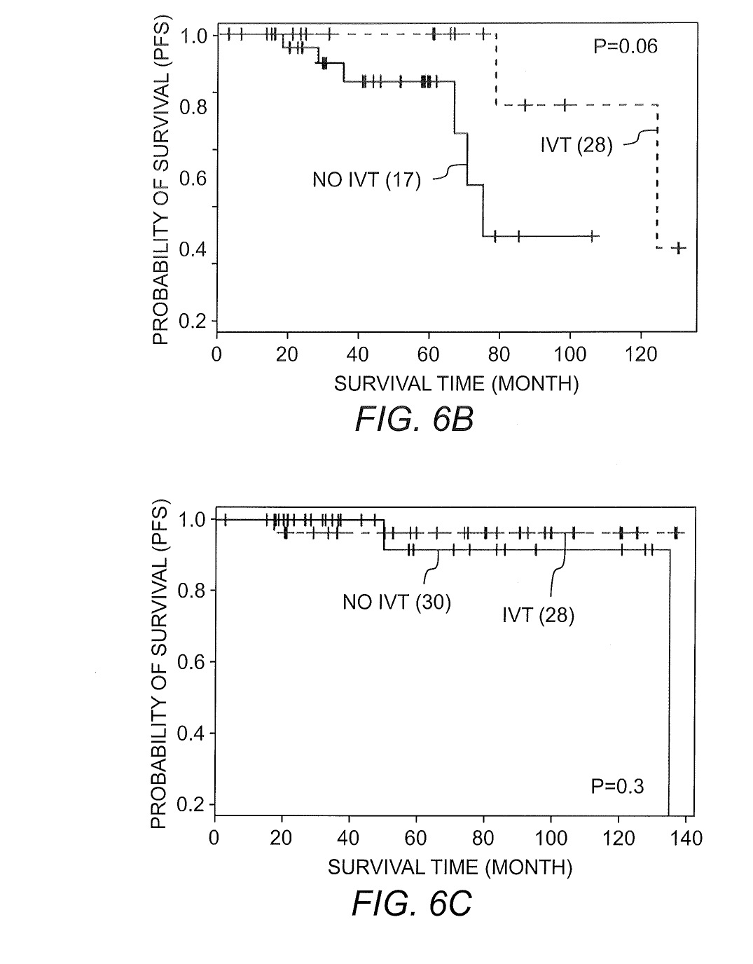

Application of E2F4 Program to Predicting Intravesical Therapy Effectiveness in NMIBC. Some of the NMIBC samples in GSE13507 dataset received one cycle of intravesical BCG immunotherapy (IVT). A comparison between the IVT-treated and the IVT-untreated groups showed significantly longer progression-free survival times of the former group, indicating that, overall, NMIBC patients can benefit from IVT (FIG. 6A). It was then determined whether the E2F4 signature could be used to predict the treatment effect of IVT. Specifically, the NMIBC patients were stratified into E2F4>0 and E2F4<0 groups. Survival analyses indicated that IVT can extend the survival times of the patients in the E2F4>0 group (FIG. 6B). For the E2F4<0 group, all patients showed good prognosis with or without IVT (FIG. 6C). Thus, applying IVT treatment to this group may not benefit patients. Considering the possible harm and risk of the treatment, this analysis indicated that patients with E2F4 should not be treated by IVT. Thus, the E2F4 program is of use as a predictive marker for determining whether IVT should be applied to a NMIBC patient.

E2F4 Scores in Bladder Cancer Molecular Subtypes. Based on gene expression profile, bladder tumor samples can be classified into five different molecular subtypes: urobasal A, genomically unstable, urobasal B, squamous cell carcinoma-like (SCC-like), and an infiltrated class of tumors (Darnell, Jr. (2002) Nat. Rev. Cancer 2:740-9). These molecular subtypes showed distinct survival patterns. The E2F4 scores were calculated for samples from the GSE32894 dataset, in which the molecular subtypes of samples were carefully defined. It was observed that the urobasal A samples tended to have lower E2F4 scores, consistent with their good prognosis, whereas the SCC-like samples had the highest E2F4 scores, which was known to be associated with poor prognosis. The other subtype with poor prognosis, urobasal B, also showed relatively high E2F4 scores. The infiltrated subtype showed intermediate prognosis, and samples of this subtype had intermediate E2F4 scores. The genomically unstable subtype was associated with intermediate prognosis; however, it was found that samples of this subtype tended to have high E2F4 scores. This indicated that prognosis was not fully determined by the proliferation of cells captured by the E2F4 program. It was also affected by some other factors such as genome stability.

Validation of E2F4 Program in Meta-Bladder Cancer Datasets. To validate the findings obtained from the GSE13507 dataset, the effectiveness of the E2F4 program for progression prediction in two meta-bladder datasets was investigated. The two meta-datasets were created by combining the previously published bladder cancer gene expression data with matched survival information for patients. In order to include as many samples as possible, the overall survival time was examined, for which data were available for the majority of samples. In the first meta-dataset, gene expression was measured using one-channel arrays and was composed of 482 samples from three independent studies. In the second meta-dataset, gene expression was measured using two-channel arrays and was composed of 240 samples from two independent studies.

The capacity of the E2F4 program for predicting the overall survival time of patients was validated in both meta-datasets. In the one-channel metadata, the E2F4>0 group showed significantly shorter survival times than the E2F4<0 groups with P=4E-9 (log-rank test). Similarly, in the two-channel metadata the high-E2F4 group showed significantly shorter survival times than the low-E2F4 groups with P=6E-11 (log-rank test). When samples were further divided into NMIBC (superficial) and MIBC (invasive), it was observed that the E2F4 program was more effective for predicting overall survival of NMIBC than MIBC samples. The program resulted in P=0.04 and P=0.02 in the NMIBC and MIBC meta-datasets, respectively. For MIBC samples, the two groups stratified based on E2F4 scores were not significantly different in their overall survival times (P=0.2 and P=0.09 respectively). This was caused by the fact that (i) overall survival time is less bladder cancer related than progression-free survival time, and thus more difficult to predict; and (ii) some MIBC patients have been treated by chemotherapy, which complicates the analysis. It should be noted that due to the majority of samples in the two-channel metadata having negative E2F4 scores, samples were stratified for this dataset using the median E2F4 scores as the threshold. In all the two-channel arrays used in this analysis, RNA pooled from ten human cell lines was used as the reference. Thus, negative E2F4 scores indicated relatively lower E2F4 activities in bladder tumors with respect to the pooled RNA reference.

### EXAMPLE 4

### Refined Signatures for Calculating E2F4 Activity

The methodology described in Example 1 calculates E2F4 score in samples based on genome-wide gene expression profiles. Namely, the expression levels of all genes need to be quantified simultaneously. However, for clinical applications, this is not practical. Therefore, the E2F4 signature was further refined to develop a prognostic model that is more amenable to clinical translation into a cost-effective assay that is easy to perform. Specifically, only a subset of E2F4 target genes that were most highly correlated with E2F4 score in terms of their expression were selected and used to estimate the E2F4 activity in cancer samples. That is, E2F4 activity was calculated based solely on the core set of highly informative target genes, and therefore the expression of these minimal set of genes can be quantified in the genomic assay.

The E2F4 scores in TCGA (The Cancer Genome Atlas) bladder cancer samples was calculated by BASE, and the top E2F4 target genes that were most correlated with E2F4 scores in their expression were selected to define a multi-gene signature. Subsequently, the expression level of these genes in TCGA bladder cancer data was analyzed using principle component analysis (PCA) to obtain the first principle component (PC1). Since the selected genes were all highly correlated with E2F4 score, PC1 was highly correlated with E2F4 score and thus could used to estimate E2F4 activity in patient samples. Based on the PCA result in TCGA bladder cancer data, an estimated E2F4 score (denoted as PES, PCA-derived E2F4 score) was defined as the linear combination of the p genes:

PES=Σᵢ₌₁^{p}βᵢeᵢ,

where “βᵢ” is the loading of gene “i” for PC1, and “eᵢ” is the expression level of gene “i” in the sample. Given a bladder sample, the PES can be calculated from the equation based on the expression levels of the p genes and used to estimate E2F4 activity.

Using this method, a 22-gene signature was identified for predicting breast cancer prognosis (Table 6) prediction, and a 33-gene signature for bladder cancer prognostic prediction (Table 7). The control genes used in these analyses included ACTB, GAPDH, RPLPO, GUSB, and TFRC. However, a combination of the following genes can also be used as control genes: ACTS, B2M, GAPD, HMBS, HPRT1, RPL13A, SDHA, TBP, UBC and YWHAZ. See, Vandesompele, et al (2002) Genome Biology 3:research0034-research0034.11.

TABLE 6

| | | HGNC |
|---|---|---|
| Gene | Description | Acc. No. |
| FOXM1 | forkhead box M1 | 3818 |
| PRC1 | protein regulator of cytokinesis 1 | 9341 |
| NCAPG | non-SMC condensin I complex, subunit G | 24304 |
| KIF2C | kinesin family member 2C | 6393 |
| BUB1B | budding uninhibited by benzimidazoles 1 | 1149 |
| | homolog beta (yeast) | |
| NUSAP1 | nucleolar and spindle associated protein 1 | 18538 |
| BUB1 | budding uninhibited by benzimidazoles 1 | 1148 |
| | homolog (yeast) | |
| TRIP13 | thyroid hormone receptor interactor 13 | 12307 |
| KIF20A | kinesin family member 20A | 9787 |
| CENPA | centromere protein A | 1851 |
| BIRC5 | baculoviral IAP repeat-containing 5 | 593 |
| CEP55 | centrosomal protein 55 kDa | 1161 |
| CDK1 | cell division cycle 2, G1 to S and G2 to M | 1722 |
| ASPM | asp (abnormal spindle) homolog, | 19048 |
| | microcephaly associated (Drosophila) | |
| ZWINT | ZW10 interactor | 13195 |
| CENPF | centromere protein F, 350/400 ka (mitosin) | 1857 |
| NDC80 | NDC80 homolog, kinetochore complex | 16909 |
| | component (S. cerevisiae) | |
| CDCA8 | cell division cycle associated 8 | 14629 |
| NEK2 | NIMA (never in mitosis gene a)-related | 7745 |
| | kinase 2 | |
| MAD2L1 | MAD2 mitotic arrest deficient-like 1 | 6763 |
| | (yeast) | |
| MKI67 | antigen identified by monoclonal antibody | 7107 |
| | Ki-67 | |
| CDCA3 | cell division cycle associated 3 | 14624 |

HGNC, HUGO Gene Nomenclature Committee.

TABLE 7

| | | HGNC |
|---|---|---|
| Gene | Description | Acc. No. |
| ANLN | anillin, actin binding protein | 14082 |
| ASF1B | ASF1 anti-silencing function 1 homolog | 20996 |
| | B (S. cerevisiae) | |
| ASPM | asp (abnormal spindle) homolog, | 19048 |
| | microcephaly associated (Drosophila) | |
| AURKB | aurora kinase B | 11390 |
| BUB1 | budding uninhibited by benzimidazoles 1 | 1148 |
| | homolog (yeast) | |
| BUB1B | budding uninhibited by benzimidazoles 1 | 1149 |
| | homolog beta (yeast) | |
| CDCA3 | cell division cycle associated 3 | 14624 |
| CDCA5 | cell division cycle associated 5 | 14626 |
| CDCA8 | cell division cycle associated 8 | 14629 |
| CDK1 | cell division cycle 2, G1 to S and G2 to M | 1722 |
| CDT1 | chromatin licensing and DNA replication | 24576 |
| | factor 1 | |
| CENPA | centromere protein A | 1851 |
| CENPF | centromere protein F, 350/400 ka | 1857 |
| | (mitosin) | |
| CEP55 | centrosomal protein 55 kDa | 1161 |
| CKAP2L | cytoskeleton associated protein 2-like | 26877 |
| DTL | denticleless homolog (Drosophila) | 30288 |
| E2F2 | E2F transcription factor 2 | 3114 |
| IQGAP3 | IQ motif containing GTPase activating | 20669 |
| | protein 3 | |
| KIAA0101 | KIAA0101 | 28961 |
| KIF14 | kinesin family member 14 | 19181 |
| KIF15 | kinesin family member 15 | 17273 |
| KIF20A | kinesin family member 20A | 9787 |
| NCAPG | non-SMC condensin I complex, subunit G | 24304 |
| KIF2C | kinesin family member 2C | 6393 |
| NUSAP1 | nucleolar and spindle associated | 18538 |
| | protein 1 | |
| PRC1 | protein regulator of cytokinesis 1 | 9341 |
| RAD54L | RAD54-like (S. cerevisiae) | 9826 |
| SPAG5 | sperm associated antigen 5 | 13452 |
| TK1 | thymidine kinase 1, soluble | 11830 |
| TRIP13 | thyroid hormone receptor interactor 13 | 12307 |
| TROAP | trophinin associated protein (tastin) | 12327 |
| UBE2T | ubiquitin-conjugating enzyme E2T | 25009 |
| | (putative) | |
| UHRF1 | ubiquitin-like with PHD and ring finger | 12556 |
| | domains 1 | |

HGNC, HUGO Gene Nomenclature Committee.

What is claimed is:

1. A method of administering an aggressive breast cancer treatment comprising:
(a) providing a ER+ breast tumor tissue sample from a patient;
(b) measuring the expression of genes regulated by transcription factor E2F4 in the ER+ breast tumor tissue sample;
(c) inferring changes in transcription factor E2F4 activity in the ER+ breast tumor tissue sample using the measured expression in (b);
(d) comparing the inferred changes in transcription factor E2F4 activity in the ER+ breast tumor tissue sample to transcription factor E2F4 activity in a reference; and
(e) administering an aggressive breast cancer treatment to the patient when the ER+ breast tumor tissue sample has higher transcription factor E2F4 activity than in the reference.

2. The method of claim 1, wherein the expression of genes regulated by transcription factor E2F4 is performed by microarray analysis with probes specific to the genes regulated by transcription factor E2F4.

3. The method of claim 1, wherein the genes regulated by transcription factor E2F4 are listed in Table 1 or Table 6.

4. The method of claim 1, wherein the aggressive breast cancer treatment comprises chemotherapy, radiation or a combination thereof.

5. A method of administering intravesical BCG immunotherapy comprising:
(a) providing a non-muscle invasive bladder cancer sample from a patient;
(b) measuring the expression of genes regulated by transcription factor E2F4 in the non-muscle invasive bladder cancer sample;
(c) inferring changes in transcription factor E2F4 activity in the non-muscle invasive bladder cancer sample using the measured expression in (b);
(d) comparing the inferred changes in transcription factor E2F4 activity in the non-muscle invasive bladder cancer sample to transcription factor E2F4 activity in a reference; and
(e) administering intravesical BCG immunotherapy to the patient when the non-muscle invasive bladder cancer sample has higher transcription factor E2F4 activity than in the reference.

6. The method of claim 5, wherein the expression of genes regulated by transcription factor E2F4 is performed by microarray analysis with probes specific to the genes regulated by transcription factor E2F4.

7. The method of claim 5, wherein the genes regulated by transcription factor E2F4 are listed in Table 1 or Table 7.
